(19) United States
(12) Patent Application Publication
(10) Pub. No.: US 2019/0070266 A1
(43) Pub. Date: Mar. 7, 2019

(54) Liraglutide in Diabetic Foot Ulcer

(71) Applicant: Novo Nordisk A/S, Bagsvaerd (DK)

(72) Inventor: Soeren Rasmussen, Copenhagen (DK)

(21) Appl. No.: 16/081,550

(22) PCT Filed: Mar. 3, 2017
(86) PCT No.: PCT/EP2017/054986

§ 371 Date: Aug. 31, 2018

(30) Foreign Application Priority Data
Mar. 4, 2016 (EP) 16158737.3
Jun. 10, 2016 (EP) 16173916.4

(51) Int. Cl. A61K 38/26; A61K 47/10; A61K 47/02; A61P 17/02; A61P 3/10

(52) U.S. Cl. CPC A61K 38/26; A61K 47/10; A61P 3/10; A61P 17/02; A61K 47/02

(57) ABSTRACT

The present invention relates to the GLP-1 receptor agonist liraglutide for use in medicine.

The present invention relates to the GLP-1 receptor agonist liraglutide for use in diabetic foot ulcer conditions.

### BACKGROUND

Diabetes is a metabolic disorder characterized by hyperglycaemia that is associated with a high risk of cardiovascular and other serious health-related consequences. A person with diabetes is two to three times more likely to die from cardiovascular causes than people with no history of diabetes, even after controlling for other cardiovascular risk factors. They are also at very high risk of developing serious microvascular complications ultimately leading to premature death: nephropathy and renal failure, retinal disease and blindness, autonomic and peripheral neuropathy, as well as other conditions related to the vascular system: hypertension, lower limb amputation, cognitive decline, and erectile dysfunction.

The majority of people with diabetes have type 2 diabetes, which is characterised by insulin resistance and eventually impaired insulin secretion. Optimal glycaemic control is the treatment goal in subjects with type 2 diabetes, since the risk of long-term complications is increased with poor glycaemic control. Despite the availability of several oral anti-diabetic drugs and insulin, a significant proportion of subjects with type 2 diabetes do not achieve the recommended target levels. With the increasing incidence and prevalence of type 2 diabetes, there is an unmet medical need for treatment alternatives with improved efficacy, safety, and convenience.

### SUMMARY

In some embodiments the present invention relates to a method of treating type 2 diabetes, comprising administering liraglutide in a therapeutically effective amount to a subject in need thereof, wherein said subject has (i) vascular disease selected from the group consisting of cardiovascular disease, cerebrovascular disease, peripheral vascular disease, chronic renal failure, and chronic heart failure, and/or (ii) one or more risk factors of vascular disease selected from the group consisting of microalbuminuria, proteinuria, hypertension, left ventricular hypertrophy, left ventricular systolic dysfunction, left ventricular diastolic dysfunction, and ankle/brachial index <0.9; wherein said method reduces or delays diabetic foot ulcer.

### DESCRIPTION

Diabetic foot ulcer is a late stage complication of diabetes and is the primary reason for hospitalisation among late stage diabetic indications. Diabetic foot ulcers are also a major reason for diabetes-related amputations.

In some embodiments the present invention relates to a method for reducing or delaying the development of diabetic foot ulcer, comprising administering liraglutide in a therapeutically effective amount to a subject in need thereof, wherein the subject has type 2 diabetes and one or more risk factors of vascular disease.

In some embodiments the present invention relates to a method of treating type 2 diabetes, comprising administering liraglutide in a therapeutically effective amount to a subject in need thereof, wherein said subject has (i) vascular disease selected from the group consisting of cardiovascular disease, cerebrovascular disease, peripheral vascular disease, chronic renal failure, and chronic heart failure, and/or (ii) one or more risk factors of vascular disease selected from the group consisting of microalbuminuria, proteinuria, hypertension, left ventricular hypertrophy, left ventricular systolic dysfunction, left ventricular diastolic dysfunction, and ankle/brachial index <0.9; wherein said method reduces or delays diabetic foot ulcer.

In some embodiments the present invention relates to a method of treating type 2 diabetes, comprising administering liraglutide in a therapeutically effective amount to a subject in need thereof, wherein said subject has (i) vascular disease selected from the group consisting of cardiovascular disease, cerebrovascular disease, peripheral vascular disease, chronic renal failure, and chronic heart failure, and/or (ii) one or more risk factors of vascular disease selected from the group consisting of microalbuminuria, proteinuria, hypertension, left ventricular hypertrophy, left ventricular systolic dysfunction, left ventricular diastolic dysfunction, and ankle/brachial index <0.9; wherein said method a) reduces or delays diabetic foot ulcer; b) reduces or delays serious diabetic foot ulcer; c) reduces or delays severe or moderate diabetic foot ulcer; and/or d) reduces or delays severe diabetic foot ulcer.

In some embodiments the method reduces or delays severe or moderate diabetic foot ulcer. In some embodiments the method reduces or delays severe diabetic foot ulcer. In some embodiments severity of a diabetic foot ulcer may be classified as severe (ulcer penetrating to bone or joint), moderate (ulcer penetrating through tendon or capsule), or mild (superficial ulcer not penetrating tendon, bone, or joint). In some embodiments a severe diabetic foot ulcer is present when the diabetic foot ulcer results in considerable interference with the subject's daily activities, and is unacceptable. In some embodiments a moderate diabetic foot ulcer is present when the diabetic foot ulcer results in marked symptoms, moderate interference with the subject's daily activities. In some embodiments a mild diabetic foot ulcer is present when the diabetic foot ulcer results in no or transient symptoms, no interference with the subject's daily activities.

In some embodiments the method reduces or delays serious diabetic foot ulcer. In some embodiments degree of a diabetic foot ulcer may be classified as either serious or non-serious, for example depending on its surface area, depth and location. In some embodiments a serious diabetic foot ulcer is a foot ulcer which results in the subject experiencing at least one event selected from the group consisting of (i) death; (ii) a life-threatening experience; (iii) hospitalisation or prolongation of existing hospitalisation; (iv) a persistent or significant disability/incapacity; (v) important medical events that may not result in death, be life-threatening, or require hospitalisation when, based upon appropriate medical judgement, they may jeopardise the subject and may require medical or surgical intervention to prevent one of the outcomes (i)-(iv) listed in this definition. In some embodiments suspicion of transmission of infectious agents is also a serious diabetic foot ulcer. A non-serious diabetic foot ulcer is a diabetic foot ulcer which does not fulfil the definition of a serious diabetic foot ulcer. In some embodiments the term “life-threatening” as used herein refers to an event in which the subject was at risk of death at the time of the event. It does not refer to an event which hypothetically might have caused death if it was more severe. In some embodiments a subject is considered to be hospitalised (i) when admitted to a hospital (irrespective of the duration of physical stay), or (ii) when not admitted to a hospital, but stays at the hospital for treatment or observation for more than 24 hours. In some embodiments a subject is not considered to be hospitalised when hospitalised for (i) administrative, trial related and social purposes, or (ii) surgical procedures planned prior to starting administration of liraglutide. In some embodiments the term “disability/incapacity” as used herein means that following the event the subject or clinical investigation subject has significant, persistent or permanent change, impairment, damage or disruption in his body function or structure, physical activity and/or quality of life. In some embodiments the term “important medical events” as used herein means events which may jeopardise the subject or require intervention to prevent a seriousness criterion, for example adverse events which suggest a significant hazard or puts the subject or clinical investigation subject at risk, such as drug-interactions, contra-indications or precautions, occurrence of malignancies or development of drug dependency or drug abuse. In some embodiments a serious diabetic foot ulcer is a foot ulcer which results in death of the subject, also referred to herein as fatal outcome. In some embodiments a serious diabetic foot ulcer is a foot ulcer which results in the subject experiencing a life-threatening experience. In some embodiments a serious diabetic foot ulcer is a foot ulcer which results in the subject experiencing hospitalisation or prolongation of existing hospitalisation. In some embodiments a serious diabetic foot ulcer is a foot ulcer which results in the subject experiencing a persistent or significant disability/incapacity. The terms “subject” and “patient” may be used interchangeably herein.

In some embodiments the method further reduces or delays a fatal outcome for said subject.

In some embodiments the method further reduces or delays recovery with sequelae. In some embodiments the outcome of a diabetic foot ulcer may be classified as fatal, not recovered, recovered with sequelae, recovering, or recovered. The term “recovered with sequelae” may refer to the subject having recuperated but retained one or more pathological conditions resulting from the diabetic foot ulcer, for example an amputation. In some embodiments the term “recovered with sequelae” refers to the patient having recovered from the diabetic foot ulcer, but with a lasting effect due to a disease, injury, treatment or procedure caused by the diabetic foot ulcer.

In some embodiments the method comprises at least 16 months of chronic administration of liraglutide. In some embodiments the method comprises at least 14 months of chronic administration of liraglutide. In some embodiments the method comprises at least 20 months of chronic administration of liraglutide.

In some embodiments “reduces or delays” when used herein with reference to the method of the invention is “reduces the risk of”.

### Subject and Subpopulations

The subject to be administered liraglutide according to the present invention may be human, such as an adult human.

The subject to receive liraglutide administration according to the methods of the present invention has type 2 diabetes and has (i) vascular disease selected from the group consisting of cardiovascular disease, cerebrovascular disease, peripheral vascular disease, chronic renal failure, or chronic heart failure, and/or (ii) one or more risk factors of vascular disease. In some embodiments the subject has type 2 diabetes and cardiovascular disease, cerebrovascular disease, peripheral vascular disease, chronic renal failure, or chronic heart failure. The subject may have type 2 diabetes and cardiovascular disease. The subject may have type 2 diabetes and cerebrovascular disease. The subject may have type 2 diabetes and peripheral vascular disease. The subject may have type 2 diabetes and chronic renal failure. The subject may have type 2 diabetes and chronic heart failure. In some embodiments the subject has type 2 diabetes and one or more risk factors of vascular disease. These vascular diseases may be referred to as concomitant, i.e. one or more vascular diseases are present in the subject at the same time as type 2 diabetes.

In some embodiments the subject is at least 50 years of age, such as at least 60 years of age.

In some embodiments the subject has HbA_{1c} of at least 7.0%, e.g. prior to receiving liraglutide administration.

In some embodiments the subject is, except for liraglutide, anti-diabetic drug naive or treated with one or more oral anti-diabetic drugs (OADs) or treated with human NPH insulin or long-acting insulin analogue or premixed insulin, alone or in combination with OAD(s). The subject may be anti-diabetic drug naive. The subject may be treated with one or more oral anti-diabetic drugs (OADs). The subject may be treated with human NPH insulin or long-acting insulin analogue or premixed insulin, alone or in combination with OAD(s). In some embodiments the OAD may be selected from the group consisting of sulfonylureas, insulin secretagogues, thiazolidinediones, alpha-glucosidase inhibitors, dipeptidyl peptidase-4 inhibitors, sodium-glucose co-transporter-2 inhibitors, and combinations thereof. In some embodiments the OAD is sulfonylurea (e.g. glimepiride, glipizide, glyburide). In some embodiments the OAD is insulin secretagogues (e.g. biguanides such as metformin or meglitinides such as nateglinide). In some embodiments the OAD is thiazolidinediones (e.g. pioglitazone, rosiglitazone). In some embodiments the OAD is alpha-glucosidase inhibitors (e.g. acarbose, miglitol, voglibose). In some embodiments the OAD is sodium-glucose co-transporter-2 inhibitors (e.g. dapagliflozin, canagliflozin, empagliflozin). In some embodiments the OAD is dipeptidyl peptidase-4 inhibitors (e.g. sitagliptin). In some embodiments the OAD is not a dipeptidyl peptidase-4 inhibitor.

In some embodiments the subject (i) is at least 50 years of age and has cardiovascular disease, cerebrovascular disease, peripheral vascular disease, chronic renal failure, or chronic heart failure, or (ii) is at least 60 years of age and has one or more risk factors of vascular disease.

In some embodiments the subject a) (i) is at least 50 years of age and has vascular disease selected from the group consisting of cardiovascular disease, cerebrovascular disease, peripheral vascular disease, chronic renal failure, or chronic heart failure, or (ii) is at least 60 years of age and has risk factors of vascular disease; b) has HbA_{1c} of at least 7.0%, e.g. at the time prior to receiving liraglutide administration; and c) is anti-diabetic drug naive or treated with one or more oral anti-diabetic drugs (OADs) or treated with human NPH insulin or long-acting insulin analogue or premixed insulin, alone or in combination with OAD(s).

The cardiovascular disease, cerebrovascular disease, peripheral vascular disease, chronic renal failure, and/or chronic heart failure may be selected from the group consisting of: a) prior myocardial infarction; b) prior stroke or prior transient ischaemic attack (TIA); c) prior coronary, carotid or peripheral arterial revascularisation; d) >50% stenosis on angiography or other imaging of coronary, carotid or lower extremity arteries; e) history of symptomatic coronary heart disease documented by positive exercise stress test or any cardiac imaging, or unstable angina pectoris with ECG (electrocardiogram) changes; f) asymptomatic cardiac ischemia documented by positive nuclear imaging test or exercise test or dobutamine stress echo; g) chronic heart failure NYHA class and h) chronic renal failure, having clinically reached a stage corresponding to a glomerular filtration rate <60 mL/min/1.73 m² per Modification of Diet in Renal Disease (MDRD) or <60 mL/min per Cockroft-Gault formula. In some embodiments, the subject experienced the a) myocardial infarction; b) stroke or transient ischaemic attack (TIA); or c) coronary, carotid or peripheral arterial revascularisation as a prior event before the time of liraglutide administration.

The glomerular filtration rate per MDRD may be as defined in formula Ia: GFR (mL/min/1.73 m²)=175×(S_{cr})^{-1.154}×(Age)^{-0.203}×(0.742 if female)×(1.212 if African American) [Ib].

The “Cockroft-Gault formula” may be as defined by Formula Ib: CrCl (mL/min)=(N×[140−age (years)]×weight*(kg))/Serum creatinine (μM) [Ib], wherein CrCl is the Cockcroft and Gault creatinine clearance, wherein N is 1.23 for males and 1.04 for females, and wherein if actual weight is greater than 120% IBW then weight is the ideal body weight (IBW) as defined in Formula II: IBW (kg)=(no of inches over 5 ft×2.3)+M [II], wherein M is 50 for males and 45.5 for females.

Heart failure exists in different degrees of severity. The most commonly used classification system of heart failure is the New York Heart Association Functional Classification (also referred to as “NYHA”). NYHA categorises subjects in one of four classes I-IV (Table A), based on their degree of limitation during physical activity, and optionally an additional subgroup A-D based on objective assessments, for further details see The Criteria Committee of the New York Heart Association. Nomenclature and Criteria for Diagnosis of Diseases of the Heart and Great Vessels. 9th ed. Boston, Mass.: Little, Brown & Co; 1994:253-256).

TABLE A
NYHA class I-IV criteria

| NYHA | |
|---|---|
| Class | Functional Capacity of the subject |
| I | Subjects with cardiac disease but without resulting limitation of |
| | physical activity. Ordinary physical activity does not cause |
| | undue fatigue, palpitation, dyspnea, or anginal pain. |
| II | Subjects with cardiac disease resulting in slight limitation of |
| | physical activity. They are comfortable at rest. Ordinary physical |
| | activity results in fatigue, palpitation, dyspnea, or anginal pain. |
| III | Subjects with cardiac disease resulting in marked limitation of |
| | physical activity. They are comfortable at rest. Less than |
| | ordinary activity causes fatigue, palpitation, dyspnea, or anginal |
| | pain. |
| IV | Subjects with cardiac disease resulting in inability to carry on |
| | any physical activity without discomfort. Symptoms of heart |
| | failure or the anginal syndrome may be present even at rest. If |
| | any physical activity is undertaken, discomfort is increased. |

The cardiovascular disease, cerebrovascular disease, peripheral vascular disease, chronic renal failure, and/or chronic heart failure may be myocardial infarction. The cardiovascular disease, cerebrovascular disease, peripheral vascular disease, chronic renal failure, and/or chronic heart failure may be stroke or prior transient ischaemic attack (TIA). The cardiovascular disease, cerebrovascular disease, peripheral vascular disease, chronic renal failure, and/or chronic heart failure may be coronary, carotid or peripheral arterial revascularisation. The cardiovascular disease, cerebrovascular disease, peripheral vascular disease, chronic renal failure, and/or chronic heart failure may be >50% stenosis on angiography or other imaging of coronary, carotid or lower extremity arteries. The cardiovascular disease, cerebrovascular disease, peripheral vascular disease, chronic renal failure, and/or chronic heart failure may be history of symptomatic coronary heart disease documented by positive exercise stress test or any cardiac imaging, or unstable angina pectoris with ECG changes. The cardiovascular disease, cerebrovascular disease, peripheral vascular disease, chronic renal failure, and/or chronic heart failure may be asymptomatic cardiac ischemia documented by positive nuclear imaging test or exercise test or dobutamine stress echo. The cardiovascular disease, cerebrovascular disease, peripheral vascular disease, chronic renal failure, and/or chronic heart failure may be chronic heart failure NYHA class II-III. The cardiovascular disease, cerebrovascular disease, peripheral vascular disease, chronic renal failure, and/or chronic heart failure may be chronic renal failure, having clinically reached a stage corresponding to a glomerular filtration rate <60 mL/min/1.73 m² per Modification of Diet in Renal Disease (MDRD) or <60 mL/min per Cockroft-Gault formula.

The “risk factors of vascular disease” (also referred to herein as “other specified risk factors of vascular disease”) may be selected from the group consisting of microalbuminuria, proteinuria, hypertension, left ventricular hypertrophy, left ventricular systolic dysfunction, left ventricular diastolic dysfunction, and ankle/brachial index <0.9. In some embodiments the “risk factors of vascular disease” may be selected from the group consisting of a) microalbuminuria or proteinuria; b) hypertension and/or left ventricular hypertrophy by ECG or imaging; c) left ventricular systolic or diastolic dysfunction by imaging; and d) ankle/brachial index <0.9. The “risk factors of vascular disease” may be microalbuminuria or proteinuria. The “risk factors of vascular disease” may be hypertension and/or left ventricular hypertrophy by ECG or imaging. The “risk factors of vascular disease” may be left ventricular systolic or diastolic dysfunction by imaging. The “risk factors of vascular disease” may be ankle/brachial index <0.9.

### BMI

In some embodiments the subject has a BMI of at least 30 kg/m². BMI (body mass index) is a measure of body fat based on height and weight. The formula for calculation is BMI=(weight in kilograms)/(height in meters)². In some embodiments the subject has a BMI in the range of 30-50 kg/m². In some embodiments the subject has a BMI of at least 33 kg/m². In some embodiments the subject has a BMI of at least 35 kg/m². In some embodiments the subject has a BMI of at least 37 kg/m². In some embodiments the subject has a BMI of at least 40 kg/m². In some embodiments the subject has a BMI of up to 45 kg/m². In some embodiments the subject has a BMI of up to 40 kg/m².

### Exclusion Criteria

In some embodiments the subject does not have type 1 diabetes. In some embodiments the subject does not receive administration of a GLP-1 receptor agonist (exenatide or other) or pramlintide or any dipeptidyl peptidase 4 (DPP-4) inhibitor prior to initiating administration of liraglutide according to the present invention. In some embodiments the subject does not receive administration of insulin other than insulin selected from the group consisting of human neutral protamine hagedorn (NPH) insulin, long-acting insulin analogue or premixed insulin. In some embodiments, and in connection with intercurrent illness, the subject receives short-term administration of insulin other than insulin selected from the group consisting of human NPH insulin, long-acting insulin analogue or premixed insulin. Acute decompensation of glycaemic control requiring immediate intensification of treatment to prevent acute complications of diabetes (e.g., diabetic ketoacidosis) in the previous 3 months. In some embodiments the subject does not have an acute coronary or cerebrovascular event in the previous 14 days. In some embodiments the subject does not receive continuous renal replacement therapy. In some embodiments the subject does not have end-stage liver disease. In some embodiments the subject does not have chronic heart failure NYHA IV. In some embodiments the subject does not have a prior solid organ transplant or awaiting solid organ transplant. In some embodiments the subject does not have family or personal history of multiple endocrine neoplasia type 2 (MEN2) or familial medullary thyroid carcinoma (FMTC). In some embodiments the subject does not have personal history of non-familial medullary thyroid carcinoma. In some embodiments the subject does not have malignant neoplasm requiring chemotherapy, surgery, radiation or palliative therapy in the previous 5 years. In some embodiments the subject has intraepithelial squamous cell carcinoma of the skin (Bowen's disease) treated with topical 5-fluorouracil (5FU) and subjects with basal cell skin cancer.

### Liraglutide

Liraglutide is the GLP-1 receptor agonist Arg34,Lys26-(N-epsilon-(gamma-L-glutamyl(N-alfa-hexadecanoyl)))-GLP-1(7-37). Liraglutide may be prepared as described in Example 37 of WO98/08871.

### Pharmaceutical Composition

Liraglutide may be administered in the form of a pharmaceutical composition. The pharmaceutical composition may comprise liraglutide in a concentration from 0.1 mg/ml to 100 mg/ml. In some embodiments the pharmaceutical composition comprises 0.01-50 mg, or 0.01-20 mg, or 0.01-10 mg/ml liraglutide. In some embodiments the pharmaceutical composition comprises 1-20 mg/ml liraglutide.

The pharmaceutical composition may further comprise one or more pharmaceutically acceptable excipients, for example selected from the group consisting of buffer system, preservative, tonicity agent, chelating agent, stabilizer and surfactant. In some embodiments the pharmaceutical composition comprises one or more pharmaceutically acceptable excipients, such as one or more selected from the group consisting of a buffer, an isotonic agent, and a preservative. The formulation of pharmaceutically active ingredients with various excipients is known in the art, see e.g. Remington: The Science and Practice of Pharmacy (e.g. 19th edition (1995), and any later editions). The term “excipient” broadly refers to any component other than the active therapeutic ingredient(s), e.g. liraglutide. The excipient may be an inert substance, an inactive substance, and/or a not medicinally active substance.

In some embodiments the pharmaceutical composition comprises a phosphate buffer, such as a sodium phosphate buffer, e.g. disodium phosphate. In some embodiments the pharmaceutical composition comprises an isotonic agent, such as propylene glycol. In some embodiments the pharmaceutical composition comprises a preservative, such as phenol.

The pharmaceutical composition may be in the form of a solution or a suspension. In some embodiments the pharmaceutical composition is aqueous composition, such as an aqueous solution or an aqueous suspension. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water. An aqueous composition may comprise at least 50% w/w water, or at least 60%, 70%, 80%, or even at least 90% w/w of water. In some embodiments the pharmaceutical composition has a pH in the range of 7.5-9.0.

In some embodiments liraglutide is administered in the form of a pharmaceutical composition comprising about 1-20 mg/ml liraglutide, about 2-15 mM phosphate buffer, about 2-25 mg/ml propylene glycol, about 1-18 mg/ml phenol, and has a pH in the range of 7.5-9.0. In some embodiments liraglutide is administered in the form of a pharmaceutical composition comprising about 6 mg/ml liraglutide, about 1.42 mg/ml disodium phosphate dihydrate, about 14.0 mg/ml propylene glycol, about 5.5 mg/ml phenol, and has pH of about 8.15. In some embodiments liraglutide is administered in the form of a pharmaceutical composition comprising 6 mg/ml liraglutide, 1.42 mg/ml disodium phosphate dihydrate, 14.0 mg/ml propylene glycol, 5.5 mg/ml phenol, and has pH of 8.15.

### Administration Regimen

Liraglutide may be administered in a therapeutically effective amount, such as an amount therapeutically effective to treat type 2 diabetes. The therapeutically effective amount of liraglutide can be assessed by a medical doctor. The dosage of liraglutide may be in the range from 0.1 to 10 mg.

Liraglutide may be administered once daily. In some embodiments liraglutide is administered once daily at any time in the day. In some embodiments the daily dosage of liraglutide is in the range from 0.4 to 4.0 mg, such as in the range from 0.4 to 2.0 mg. In some embodiments the daily dosage of liraglutide is selected from the group consisting of 0.6, 1.2, and 1.8 mg. In some embodiments the daily dosage of liraglutide is 3.0 mg.

In some embodiments the term “chronic treatment” as used herein with reference to liraglutide means administration in an amount and frequency to provide a therapeutic effect. In some embodiments the term “chronic treatment” as used herein with reference to liraglutide means once daily administration 0.4-4.0 mg, such as 0.6, 1.2, or 1.8 mg, liraglutide.

Liraglutide may be administered via parenteral administration, for example subcutaneous injection. Liraglutide may be administered using a pen-injector, such as a 3 ml disposable pen-injector.

Unless otherwise states, ranges herein include their end points. In some embodiments the term “a” means “one or more”. In some embodiments, and unless otherwise indicated in the specification, terms presented in singular form also include the plural situation. Herein the term “about” means±10% of the value referred to, and includes the value.

### Examples

### List of Abbreviations

MACE: Major adverse cardiovascular event

HbA_{1c}: Glycosylated haemoglobin

GLP-1: Glucagon-like peptide-1

BMI: Body mass index

CV: cardiovascular

OAD: Oral antidiabetic drug

TIA: transient ischaemic attack

CI: Confidence interval

### Trial: Materials and Methods

A long-term, multi-centre, international, randomised double-blind, placebo-controlled trial with 9340 human subjects was carried out with treatment for at least 3.5 years and up to 5 years per subject; and this trial concerned the incidence of cardiovascular events in adult human subjects with type 2 diabetes that were at high risk for cardiovascular events, including such subjects with existing cardiovascular disease. The primary objective of this trial was to determine the long term effect of treatment with liraglutide compared to placebo on cardiovascular events in subjects with type 2 diabetes. The secondary objective was to assess the efficacy and safety with regard to clinically important events or other surrogate parameters of treatment with liraglutide compared to placebo in adults with type 2 diabetes that were at high risk for cardiovascular events. All trial endpoints were collected and assessed throughout the entire duration of the trial. Subject inclusion and exclusion criteria were as described in Table 2. The subject's characteristics, cardiovascular risk profile, renal function, cardiovascular medication, and antidiabetic treatment regimens of the randomised subjects at baseline were as shown in Tables 3a-e. Overall trial duration was planned as 18 months of recruitment period followed by 42 months of treatment from last subject randomised. The trial started with an open-label run-in period of two weeks with placebo following which subjects were randomised in a 1:1 manner to receive liraglutide or placebo as an add-on to their standard of care (SOC) treatment. The subject's SOC treatment was as shown in Table 4. After randomisation, treatment with liraglutide or placebo was double-blind throughout the trial. Subjects were started on 0.6 mg of liraglutide or placebo. The term “placebo” as used herein refers to a formulation identical to the liraglutide formulation except not comprising liraglutide and the placebo was administered in the volume used in the equivalent liraglutide dosage. Dose escalation of liraglutide or placebo proceeded to 1.2 mg after one week followed by dose escalation to 1.8 mg after one week. After the dose escalation, 95% of subjects received 1.8 mg of liraglutide or placebo, 5% of subjects received 1.2 mg of liraglutide or placebo, and 5% of subjects received 0.6 mg of liraglutide or placebo. Dose increase period could be extended if required in view of a subject's tolerance to the trial product (i.e. liraglutide or placebo). The dosage could be reduced at any time in the trial if required by the subject's tolerance to the trial product. Subjects received liraglutide or placebo by subcutaneous administrations once daily in addition to the subject's standard treatment at a maximum dose of 1.8 mg liraglutide or placebo. The subcutaneous injection was made either in the abdomen, thigh or upper arm. The formulations were administered in the form of an aqueous solution comprising liraglutide or placebo, both using a 3 ml disposable pen-injector. This pen-injector was identical for the liraglutide and placebo administrations. This aqueous solution contained 6.0 mg/ml liraglutide, 1.42 mg/ml disodium phosphate dihydrate, 14.0 mg/ml propylene glycol, 5.5 mg/ml phenol, and had pH 8.15. Liraglutide may be prepared as described in WO98/08871.

The term “baseline” herein (e.g. used as part of “baseline characteristics” or “baseline cardiovascular risk profile”) may refer to the level of a certain parameter (e.g. level of HbA1c) by the determination made in connection with the medical visit at the time of randomisation of the subject. In some embodiments the term baseline refers to a parameter before initiating administration of liraglutide, e.g. the history of a certain event in a subject.

The results of this trial may be presented herein as a number or fraction of subjects experiencing an event. Alternatively, the results of this trial may be presented with hazard ratios estimated in a Cox proportional hazard model, which is the standard statistical model used for estimating time to an event. The term “hazard ratio” (also referred to as “HR”) as used herein means the instantaneous risk ratio of experiencing an event when administered liraglutide compared to placebo which are the two treatments in this trial. An upper limit of the 95% confidence interval (CI) for the HR of less than 1.00 means that the estimated treatment ratio between liraglutide and placebo with respect to the event of interests is statistically significant in favour of liraglutide on a 5% significance level. A 5% significance level is the standard level for investigating significance in clinical trials. For example, a HR value of 0.78 for time to first CV death with a 95% CI of (0.66; 0.94) means that liraglutide provides an estimated 22% risk reduction of experiencing CV death at any given point in time compared to placebo and this risk reduction is statistically significant because 0.94 is less than 1.00.

TABLE 2
Subject inclusion and exclusion criteria (all inclusion criteria were fulfilled for
eligible subjects; one or more exclusion criteria were fulfilled for subjects to be excluded;
however, 150 patients violated at least one inclusion or exclusion criteria)

| Inclusion Criteria | Exclusion Criteria |
|---|---|
| Men or women with type 2 diabetes | Type 1 diabetes |
| Age ≥ 50 years at screening and | Use of a GLP-1 receptor agonist |
| concomitant cardiovascular disease, | (exenatide, liraglutide or other) or |
| cerebrovascular disease, peripheral | pramlintide or any dipeptidyl |
| vascular disease, chronic renal failure, or | peptidase 4 (DPP-4) inhibitor within |
| chronic heart failure selected from the | the 3 months prior to screening (trial |
| group consisting of: a) prior myocardial | start) |
| infarction; b) prior stroke or prior | Use of insulin other than human |
| transient ischaemic attack (TIA); c) prior | neutral protamine hagedorn (NPH) |
| coronary, carotid or peripheral arterial | insulin or long-acting insulin analogue |
| revascularisation; d) >50% stenosis on | or premixed insulin within 3 months |
| angiography or other imaging of | prior to screening. Short-term use of |
| coronary, carotid or lower extremity | other insulin during this period in |
| arteries; e) history of symptomatic | connection with intercurrent illness |
| coronary heart disease documented by | was allowed, at Investigator's |
| positive exercise stress test or any | discretion |
| cardiac imaging, or unstable angina | Acute decompensation of glycaemic |
| pectoris with ECG (electrocardiogram) | control requiring immediate |
| changes; f) asymptomatic cardiac | intensification of treatment to prevent |
| ischemia documented by positive nuclear | acute complications of diabetes (e.g., |
| imaging test or exercise test or | diabetic ketoacidosis) in the previous |
| dobutamine stress echo; g) chronic heart | 3 months |
| failure NYHA class II-III; and h) chronic | An acute coronary or cerebrovascular |
| renal failure, having clinically reached a | event in the previous 14 days |
| stage corresponding to a glomerular | Current continuous renal replacement |
| filtration rate < 60 mL/min/1.73 m² per | therapy |
| Modification of Diet in Renal Disease | End-stage liver disease |
| (MDRD) or < 60 mL/min per Cockroft- | Chronic heart failure NYHA IV |
| Gault formula;; wherein “prior” refers to | A prior solid organ transplant or |
| before initiation of liraglutide | awaiting solid organ transplant |
| administration; OR age ≥ 60 years at | Family or personal history of multiple |
| screening and other specified risk factors | endocrine neoplasia type 2 (MEN2) or |
| of vascular disease selected from the | familial medullary thyroid carcinoma |
| group consisting of: a) microalbuminuria | (FMTC) |
| or proteinuria; b) hypertension and/or | Personal history of non-familial |
| left ventricular hypertrophy by ECG or | medullary thyroid carcinoma |
| imaging; c) left ventricular systolic or | Malignant neoplasm requiring |
| diastolic dysfunction by imaging; and d) | chemotherapy, surgery, radiation or |
| ankle/brachial index < 0.9 | palliative therapy in the previous 5 |
| HbA_{1c} ≥ 7.0% at screening | years. Subjects with intraepithelial |
| Anti-diabetic drug naive or treated with | squamous cell carcinoma of the skin |
| one or more oral anti-diabetic drugs | (Bowen's disease) treated with topical |
| (OADs) or treated with human NPH | 5-fluorouracil (5FU) and subjects with |
| insulin or long-acting insulin analogue or | basal cell skin cancer are allowed to |
| premixed insulin, alone or in combination | enter the trial |
| with OAD(s) | |

TABLE 3a
Baseline characteristics

| | Liraglutide | Placebo |
|---|---|---|
| Number of subjects | 4668 | 4672 |
| Male sex, N (%) | 3011 (64.5) | 2992 (64.0) |
| Age, years | 64.2 | 64.4 |
| Diabetes duration, years | 12.8 | 12.9 |
| HbA_{1c}, % | 8.7 | 8.7 |
| BMI, kg/m² | 32.5 | 32.5 |
| Body weight, kg | 91.9 | 91.6 |
| Systolic blood pressure, mmHg | 135.9 | 135.9 |
| Diastolic blood pressure, mmHg | 77.2 | 77.0 |
| Heart failure*, N (%) | 832 (17.8) | 821 (17.6) |

Full analysis set;
data are mean unless stated otherwise.
*Heart failure includes NYHA class I, II and III.
%: proportion of subjects.
BMI: body mass index.
HbA1c: glycosylated haemoglobin.
NYHA: New York Heart Association.

TABLE 3b
Cardiovascular risk profile at baseline

| | Liraglutide | | Placebo | |
|---|---|---|---|---|
| | N | % | N | % |
| Number of subjects | 4668 | | 4672 | |
| Age ≥ 50 years and established CV | 3815 | 81.7 | 3749 | 80.2 |
| disease | | | | |
| Prior myocardial infarction | 1464 | 31.4 | 1400 | 30.0 |
| Prior stroke or prior TIA | 730 | 15.6 | 777 | 16.6 |
| Prior arterial revascularisation | 1766 | 37.8 | 1719 | 36.8 |
| >50% stenosis on angiography | 1188 | 25.4 | 1191 | 25.5 |
| Documented history of symptomatic | 412 | 8.8 | 406 | 8.7 |
| coronary heart disease | | | | |
| Documented asymptomatic cardiac | 1241 | 26.6 | 1231 | 26.3 |
| ischaemia | | | | |
| Chronic heart failure NYHA II or III | 653 | 14.0 | 652 | 14.0 |
| Chronic kidney failure | 1185 | 25.4 | 1122 | 24.0 |
| Age ≥ 60 years and risk factors for CV | 853 | 18.3 | 923 | 19.8 |
| disease | | | | |
| Microalbuminuria or proteinuria | 504 | 10.8 | 560 | 12.0 |
| Hypertension and/or left ventricular | 249 | 5.3 | 253 | 5.4 |
| hypertrophy | | | | |
| Left ventricular systolic or diastolic | 204 | 4.4 | 191 | 4.1 |
| dysfunction | | | | |
| Ankle/brachial index < 0.9 | 126 | 2.7 | 134 | 2.9 |

Full analysis set.
*Chronic kidney failure was defined as having clinically reached a stage corresponding to eGFR <60 mL/min/1.73 m² per MDRD or <60 mL/min per Cockroft-Gault formula, reported at the discretion of the investigator.
%: proportion of subjects.
CV: cardiovascular.
eGFR: estimated glomerular filtration rate.
MDRD: modification of diet in renal disease.
N: number of subjects.
NYHA: New York Heart Association.
TIA: transient ischaemic attack.

TABLE 3c
Renal function at baseline

| | Liraglutide | | Placebo | |
|---|---|---|---|---|
| | N | % | N | % |
| Number of subjects | 4668 | 100.0 | 4672 | 100.0 |
| Normal renal function (eGFR ≥ 90) | 1620 | 34.7 | 1655 | 35.4 |
| Mild impairment (eGFR 60-89) | 1932 | 41.4 | 1975 | 42.3 |
| Moderate impairment (eGFR 30-59) | 999 | 21.4 | 935 | 20.0 |
| Severe impairment (eGFR < 30) | 117 | 2.5 | 107 | 2.3 |

Full analysis set.
eGFR (mL/min/1.73 m2) as per MDRD formula.
%: proportion of subjects.
eGFR: estimated glomerular filtration rate;
MDRD: modification of diet in renal disease.
N: number of subjects.

TABLE 3d
Cardiovascular medication at baseline

| | Liraglutide | | Placebo | |
|---|---|---|---|---|
| | N | % | N | % |
| Number of subjects | 4668 | | 4672 | |
| Antihypertensive therapy | 4322 | 92.6 | 4299 | 92.0 |
| Beta blockers | 2649 | 56.7 | 2524 | 54.0 |
| Calcium channel blockers | 1531 | 32.8 | 1477 | 31.6 |
| ACE inhibitors and ARB | 3898 | 83.5 | 3833 | 82.0 |
| Others | 468 | 10.0 | 452 | 9.7 |
| Diuretics | 1950 | 41.8 | 1949 | 41.7 |
| Loop diuretics | 823 | 17.6 | 833 | 17.8 |
| Others | 1405 | 30.1 | 1392 | 29.8 |
| Lipid-lowering drugs | 3554 | 76.1 | 3511 | 75.1 |
| Statins | 3395 | 72.7 | 3334 | 71.4 |
| Others | 655 | 14.0 | 676 | 14.5 |
| Platelet aggregation inhibitors | 3203 | 68.6 | 3119 | 66.8 |
| Acetylsalicylic acid | 2975 | 63.7 | 2899 | 62.1 |
| Others | 718 | 15.4 | 743 | 15.9 |
| Other anti-thrombotic medication | 309 | 6.6 | 314 | 6.7 |

Full analysis set.
83 subjects had missing initiation drug date, these were assumed to be on treatment at baseline.
%: proportion of subjects.
ACE: angiotensin converting enzyme.
ARB: angiotensin receptor blocker.
N: number of subjects.

TABLE 3e
Antidiabetic treatment regimens at baseline

| | |
|---|---|
| Liraglutide | Placebo |

| | | | |
|---|---|---|---|
| N | % | N | % |

| | | |
|---|---|---|
| Number of subjects | 4668 | 4672 |

| | | | | |
|---|---|---|---|---|
| Insulin-naive | 2633 | 56.4 | 2548 | 54.5 |
| Not on treatment* | 196 | 4.2 | 170 | 3.6 |
| OADs only | 2437 | 52.2 | 2378 | 50.9 |
| Insulin treatment | 2035 | 43.6 | 2124 | 45.5 |
| Insulin only | 361 | 7.7 | 376 | 8.0 |
| Insulin + OADs | 1674 | 35.9 | 1748 | 37.4 |

Full analysis set.
*Includes subjects not on insulin/OAD and subjects on no pharmacologic treatment.
%: proportion of subjects.
N: number of subjects.
OAD: oral antidiabetic drug.

TABLE 4
Standard of care guidelines for subjects in this trial

| Parameter | Standard of care guideline |
|---|---|
| Blood glucose | HbA1c ≤ 7.0% (individualized depending on patient). If > 7.0%, |
| | additional HbA1c measurement after 3 m. If HbA1c still > 7.0%, |
| | treatment was intensified to achieve target if appropriate. |
| Therapy | Lifestyle modifications and metformin are considered foundational |
| | therapy in most countries of this trial |
| Intensification | Add-on therapy: thiazolidinediones, sulfonylureas, α-glucosidase |
| | inhibitors, according to local labels (dipeptidyl peptidase-4 inhibitors |
| | and other incretin based therapies were not allowed). Insulin therapy |
| | should be based on local practice, including basal, basal/bolus, |
| | premix, and mealtime bolus (SIT). |
| Blood pressure | Target: 130/80 mmHg. |
| Antihypertensive | First line: ACE inhibitors or ARBs. Based on individual patient needs: |
| therapy | Ca²⁺-blockers, diuretics, others. |
| Lipid targets and | LDL < 100 mg/dL (<70 mg/dL in patients with previous |
| therapy | cardiovascular events). Statins recommended for all patients. |
| | Second line therapy at investigator discretion. |
| Antiplatelet | Aspirin or clopidogrel (if aspirin intolerant) for patients with prior |
| therapy | cardiovascular events (MI, cerebrovascular accident, or |
| | revascularization). |

### Example 1: Diabetic Foot Ulcer

Results from this trial concerning diabetic foot ulcer are shown in Table 4.

TABLE 4
Diabetic foot ulcer events and characterisation thereof

| | Liraglutide | | | | Placebo | | | |
|---|---|---|---|---|---|---|---|---|
| | N | % | E | R | N | % | E | R |
| Total number | 4668 | | | | 4672 | | | |
| of subjects | | | | | | | | |
| PYO in trial | 17819 | | | | 17736 | | | |
| Diabetic foot | 181 | 3.9 | 268 | 1.50 | 198 | 4.2 | 304 | 1.71 |
| ulcer events | | | | | | | | |

Serious

| | | | | | | | | |
|---|---|---|---|---|---|---|---|---|
| Yes | 93 | 2.0 | 141 | 0.79 | 116 | 2.5 | 146 | 0.82 |
| No | 102 | 2.2 | 127 | 0.71 | 103 | 2.2 | 158 | 0.89 |

Severity

| | | | | | | | | |
|---|---|---|---|---|---|---|---|---|
| Severe | 50 | 1.1 | 68 | 0.38 | 64 | 1.4 | 74 | 0.42 |
| Moderate | 97 | 2.1 | 125 | 0.70 | 107 | 2.3 | 143 | 0.81 |
| Mild | 57 | 1.2 | 75 | 0.42 | 60 | 1.3 | 87 | 0.49 |

Outcome

| | | | | | | | | |
|---|---|---|---|---|---|---|---|---|
| Fatal | 2 | <0.1 | 2 | 0.01 | 5 | 0.1 | 5 | 0.03 |
| Not recovered | 32 | 0.7 | 35 | 0.20 | 36 | 0.8 | 39 | 0.22 |
| Recovered with | 30 | 0.6 | 40 | 0.22 | 48 | 1.0 | 60 | 0.34 |
| sequelae | | | | | | | | |
| Recovering | 8 | 0.2 | 10 | 0.06 | 6 | 0.1 | 6 | 0.03 |
| Recovered | 134 | 2.9 | 180 | 1.01 | 131 | 2.8 | 193 | 1.09 |
| Unknown | 1 | <0.1 | 1 | <0.01 | 1 | <0.1 | 1 | <0.01 |

%: proportion of subjects.
E: number of events.
MESI: medical event of special interest.
N: number of subjects.
PYO: patient years of observation.
R: event rate per 100 observation years.

These results show that liraglutide reduced the frequency of diabetic foot ulcer compared to placebo. Compared to placebo liraglutide reduced the frequency of (i) serious diabetic foot ulcer as well as (ii) severe or moderate diabetic foot ulcer. Reduced frequency of outcomes which were either fatal or resulted in recovery with sequelae was shown with liraglutide compared to placebo.

TABLE 5
Adverse events by preferred term with frequency of at least 0.1%

| | |
|---|---|
| Liraglutide | Placebo |

| Preferred term | % | R | % | R |
|---|---|---|---|---|
| Diabetic foot | 2.8 | 1.0 | 3.3 | 1.3 |
| Skin ulcer | 0.6 | 0.2 | 0.6 | 0.2 |
| Diabetic foot infection | 0.3 | <0.1 | 0.3 | <0.1 |
| Gangrene | 0.2 | <0.1 | 0.3 | <0.1 |

Full analysis set.
%: proportion of subjects experiencing at least one event.
AE: adverse event.
MESI: medical event of special interest.
PT: preferred term.
R: rate (events per 100 PYO).
SAE: serious adverse event.
PYO: patient years of observation.

Diabetic foot ulcer events observed over time in a Kaplan-Meier plot showed that chronic administration of liraglutide for at least 16 months reduced the risk of developing diabetic foot ulcer compared to placebo.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-13. (canceled)

14. A method for reducing or delaying the development of diabetic foot ulcer, comprising administering liraglutide in a therapeutically effective amount to a subject in need thereof, wherein the subject has type 2 diabetes and one or more risk factors of vascular disease selected from the group consisting of microalbuminuria, proteinuria, hypertension, left ventricular hypertrophy, left ventricular systolic dysfunction, left ventricular diastolic dysfunction, and ankle/brachial index <0.9.

15. The method according to claim 14, wherein said method reduces or delays severe or moderate diabetic foot ulcer.

16. The method according to claim 14, wherein said method reduces or delays severe diabetic foot ulcer.

17. The method according to claim 14, wherein said method reduces or delays serious diabetic foot ulcer.

18. The method according to claim 14, wherein said method comprises at least 16 months of chronic administration of liraglutide.

19. The method according to claim 14, wherein the liraglutide is administered once daily.

20. The method according to claim 19, wherein the liraglutide is administered once daily in an amount in the range of 0.4-4.0 mg per day.

21. The method according to claim 20, wherein the liraglutide is administered at 0.6 mg per day.

22. The method according to claim 20, wherein the liraglutide is administered at 1.2 mg per day.

23. The method according to claim 20, wherein the liraglutide is administered at 1.8 mg per day.

24. The method according to claim 14, wherein the liraglutide is administered in the form of a pharmaceutical composition comprising about 1-20 mg/ml liraglutide, about 2-15 mM phosphate buffer, about 2-25 mg/ml propylene glycol, about 1-18 mg/ml phenol, and has a pH in the range of 7.5-9.0.

25. The method according to claim 24, wherein the liraglutide is administered in the form of a pharmaceutical composition comprising about 6 mg/ml liraglutide, about 1.42 mg/ml disodium phosphate dihydrate, about 14.0 mg/ml propylene glycol, about 5.5 mg/ml phenol, and has pH of about 8.15.

26. The method according to claim 25, wherein the liraglutide is administered in the form of a pharmaceutical composition comprising 6 mg/ml liraglutide, 1.42 mg/ml disodium phosphate dihydrate, 14.0 mg/ml propylene glycol, 5.5 mg/ml phenol, and has pH of 8.15.
